(19) United States
(12) Patent Application Publication
(10) Pub. No.: US 2006/0136033 A1
(43) Pub. Date: Jun. 22, 2006

(54) Coiled stent delivery system and method

(75) Inventors: George D. Hermann, Portola Valley, CA (US); Jonathan M. Olson, San Jose, CA (US); Marshall Tsuruda, San Jose, CA (US)

Correspondence Address: HAYNES BEFFEL & WOLFELD LLP, P O BOX 366, HALF MOON BAY, CA, 94019, US

(73) Assignee: VASCULAR ARCHITECTS, INC., San Jose, CA (US)

(21) Appl. No.: 11/018,563

(22) Filed: Dec. 20, 2004

(51) Int. Cl. A61F 2/06

(52) U.S. Cl. 623/1.11

(57) ABSTRACT

A coiled endoluminal prosthesis delivery assembly includes a catheter and a generally helical endoluminal prosthesis. The endoluminal prosthesis is placeable in a radially contracted state on the catheter. The assembly also comprises means for engaging proximal, intermediate and distal portions of the endoluminal prosthesis to maintain it in the first state, and means for controllably releasing the proximal, distal and intermediate portions to permit the endoluminal prosthesis to move towards a radially expanded state in a controlled manner. A method controllably releases the endoluminal prosthesis from the catheter within a hollow body structure. The method may include permitting at most 50%, and more preferably at most 25%, of the length of the endoluminal prosthesis to simultaneously move to the radially expanded state in contact with the hollow body structure.

### CROSS-REFERENCE TO OTHER APPLICATIONS

None.

### FEDERALLY SPONSORED RESEARCH OR DEVELOPMENT

None.

### BACKGROUND OF THE INVENTION

Stents, covered stents and other endoluminal prostheses are often useful for placement in various hollow body structures, such as blood vessels, including coronary arteries, iliac arteries and femoro-popiliteal arteries, the ureter, urethra, bronchus, biliary tract, gastrointestinal tract and the like, for the treatment of conditions which may benefit from the introduction of a reinforcing or protective structure and/or the introduction of a therapeutic agent within the body lumen. The prostheses will typically be placed endoluminally. As used herein, “endoluminally” will mean placement by percutaneous or cutdown procedures, wherein the prosthesis is transluminally advanced through the body lumen from a remote location to a target site in the lumen. In vascular procedures, the prostheses will typically be introduced “endovascularly” using a catheter over a guide wire under fluoroscopic, or other imaging system, guidance. The catheters and guide wires may be introduced through conventional access sites to the vascular system, such as through the femoral artery, or brachial and subclavian arteries, for access to the target site.

An endoluminal prosthesis typically comprises at least one radially expansible, usually cylindrical, body segment. By “radially expansible,” it is meant that the body segment can be converted from a small diameter configuration (used for endoluminal placement) to a radially expanded, usually cylindrical, configuration, which is achieved when the prosthesis is implanted at the desired target site. The prosthesis may be non-resilient, e.g., malleable, thus requiring the application of an internal force to expand it at the target site. Typically, the expansive force can be provided by a balloon catheter, such as an angioplasty balloon for vascular procedures. Alternatively, the prosthesis can be self-expanding. Such self-expanding structures may be provided by a temperature-sensitive superelastic material, such as Nitinol, which naturally assumes a radially expanded condition once an appropriate temperature has been reached. The appropriate temperature can be, for example, a temperature slightly below normal body temperature; if the appropriate temperature is above normal body temperature, some method of heating the structure must be used. Another type of self-expanding structure uses resilient material, such as a stainless steel or superelastic alloy, such as Nitinol, and forming the body segment so that it possesses its desired, radially-expanded diameter when it is unconstrained, e.g., released from radially constraining forces of a sheath. To remain anchored in the body lumen, the prosthesis will remain partially constrained by the lumen. The self-expanding prosthesis can be delivered in its radially constrained configuration, e.g. by placing the prosthesis within a delivery sheath or tube and retracting the sheath at the target site. Such general aspects of construction and delivery modalities are well known in the art.

The dimensions of a typical endoluminal prosthesis will depend on its intended use. Typically, the prosthesis will have a length in the range from 0.5 cm to 25 cm, usually being from about 0.8 cm to 10 cm, for vascular applications. The small (radially collapsed) diameter of cylindrical prostheses will usually be in the range from about 1 mm to 10 mm, more usually being in the range from 1.5 mm to 6 mm for vascular applications. The expanded diameter will usually be in the range from about 2 mm to 50 mm, preferably being in the range from about 3 mm to 15 mm for vascular applications and from about 25 mm to 45 mm for aortic applications.

One type of endoluminal prosthesis includes both a stent component and a covering component. These endoluminal prostheses are often called stent grafts or covered stents. A covered stent is typically introduced using a catheter with both the stent and covering in contracted, reduced-diameter states. Once at the target site, the stent and covering are expanded. After expansion, the catheter is withdrawn from the vessel leaving the covered stent at the target site. Coverings may be made of, for example, PTFE, ePTFE or Dacron® polyester.

Grafts are used within the body for various reasons; such as to repair damaged or diseased portions of blood vessels such as may be caused by injury, disease, or an aneurysm. It has been found effective to introduce pores into the walls of the graft to provide ingrowth of tissue onto the walls of the graft. With larger diameter grafts, woven graft material is often used. In small and large diameter vessels, porous fluoropolymers, such as ePTFE, have been found useful.

Coil-type stents can be wound about the catheter shaft in torqued compression for deployment. The coil-type stent can be maintained in this torqued compression condition by securing the ends of the coil-type stent in position on a catheter shaft. The ends are released by, for example, pulling on wires once at the target site. See, for example, U.S. Pat. Nos. 5,372,600 and 5,476,505. Alternatively, the endoluminal prosthesis can be maintained in its reduced-diameter condition by a sleeve; the sleeve can be selectively retracted to release the prosthesis. A third approach uses a balloon to expand the prosthesis at the target site. The stent is typically extended past its elastic limit so that it remains in its expanded state after the balloon is deflated and removed. One balloon expandable stent is the Palmaz-Schatz stent available from the Cordis Division of Johnson & Johnson. Stents are also available from Medtronic AVE of Santa Rosa, Calif. and Guidant Corporation of Indianapolis, Ind. A controlled release catheter assembly, such as disclosed in U.S. Pat. Nos. 6,238,430 and 6,248,122, may also be used to deploy a coiled prosthesis. See also U.S. Pat. No. 6,572,643.

The following patents may be of interest. U.S. Pat. No. 6,660,032 issued Dec. 9, 2003; U.S. Pat. No. 6,645,237 issued Nov. 11, 2003; U.S. Pat. No. 6,572,648 issued Jun. 3, 2003; U.S. Pat. No. 6,514,285 issued Feb. 4, 2003; U.S. Pat. No. 6,371,979 issued Apr. 16, 2002; U.S. Pat. No. 5,824,053 issued Oct. 20, 1998; U.S. Pat. No. 5,772,668 issued Jun. 30, 1998; U.S. Pat. No. 5,443,500 issued Aug. 22, 1995; U.S. Pat. No. 4,760,849 issued Aug. 2, 1988; and U.S. Pat. No. 4,553,545 issued Nov. 19, 1985. See also PCT Publication Number WO 94/22379 published Oct. 13, 1994; and PCT Publication Number WO 94/16629 published Aug. 4, 1994.

### BRIEF SUMMARY OF THE INVENTION

A first aspect of invention is directed to a coiled endoluminal prosthesis delivery assembly comprising a catheter, a generally helical endoluminal prosthesis having proximal and distal portions separated by an intermediate portion, the endoluminal prosthesis being placeable in a radially contracted, first state on the catheter. The assembly also comprises means for engaging each of the proximal, intermediate and distal portions thereby maintaining the endoluminal prosthesis in the first state, and means for controllably releasing the proximal, distal and intermediate portions to permit the endoluminal prosthesis to move towards a radially expanded, second state.

A second aspect of invention is directed to a coiled endoluminal prosthesis delivery assembly comprising a catheter comprising an outer surface, a lumen, and a number of openings extending from the outer surface to the lumen, and a release wire extending along a release wire path. The release wire path comprises internal release wire path segments defined within the lumen and external release wire path segments external of the catheter. The assembly also includes a generally helical endoluminal prosthesis having proximal and distal portions separated by an intermediate portion. The endoluminal prosthesis is maintained in a radially contracted condition by the release wire engaging the proximal, distal and intermediate portions of the endoluminal prosthesis at at least three of the external release wire path segments. The coiled endoluminal prosthesis is therefore releasable from the catheter when the release wire is moved along the release wire path.

A third aspect of the invention is directed to coiled endoluminal prosthesis delivery assembly comprising a catheter comprising a release wire lumen, the release wire lumen comprising a series of axially spaced-apart lumen segments, each lumen segment comprising an entrance and exit, and a release wire extending along a release wire path. The release wire path comprises internal release wire path segments defined by the lumen segments and external release wire path segments extending between the exit of one lumen segment and the entrance of another lumen segment. The assembly also includes a generally helical endoluminal prosthesis having proximal and distal portions separated by an intermediate portion. The endoluminal prosthesis is maintained in a radially contracted condition with the intermediate portion passing between the catheter and the release wire at least one of the external release wire path segments. The proximal and distal portions of the endoluminal prosthesis have release wire engagement parts with which the release wire releasably engages. The coiled endoluminal prosthesis is therefore releasable from the catheter when the release wire is moved along the release wire path.

A fourth aspect of the invention is directed to a coiled endoluminal prosthesis delivery assembly comprising a catheter, a release wire and a generally helical endoluminal prosthesis. The catheter comprises an outer surface, a main lumen, a release wire lumen, and a number of openings extending from the outer surface to the release wire lumen. The release wire extends along a release wire path, the release wire path comprising internal release wire path segments defined within the release wire lumen and external release wire path segments external of the catheter. The generally helical endoluminal prosthesis has proximal and distal portions separated by an intermediate portion. The endoluminal prosthesis is maintained in a radially contracted condition by the release wire engaging the endoluminal prosthesis at least two of the external release wire path segments. The coiled endoluminal prosthesis is therefore releasable from the catheter when the release wire is moved along the release wire path.

A fifth aspect of the invention is directed to a method for controllably releasing a generally helical endoluminal prosthesis from a catheter within a body lumen of a hollow body structure, the endoluminal prosthesis being mounted onto the catheter in a radially contracted, first state. The endoluminal prosthesis, carried by the catheter, is placed at a target location within a body lumen. The endoluminal prosthesis comprises proximal, distal and intermediate portions, each of which is temporarily retained in the radially contracted, first state. One or more of the distal, intermediate and proximal portions are released to move towards a radially expanded, second state in contact with the hollow body structure while maintaining the unreleased portions in the radially contracted, first state. The releasing step comprises permitting a portion of the endoluminal prosthesis to move to the radially expanded, second state in contact with the hollow body structure. Thereafter, the remaining one or ones of the proximal, distal and intermediate portions are selectively released to permit the entire endoluminal prosthesis to move to the radially expanded, second state in contact with the hollow body structure. The catheter is then removed from the body lumen. The releasing step may comprise permitting at most 50%, and more preferably at most 25%, of the length of the endoluminal prosthesis to simultaneously move to the radially expanded, second state in contact with the hollow body structure.

Various features and advantages of the invention will appear from the following description in which the preferred embodiments have been set forth in detail in conjunction with the accompanying drawings.

### BRIEF DESCRIPTION OF THE DRAWINGS

FIG. 1 is an overall view of a coiled stent delivery assembly made according to the invention;

FIG. 1A is an overall view of release wire assembly of FIG. 1;

FIG. 2 is a side view of the distal portion of the catheter of FIG. 1;

FIG. 3 is an enlarged overall view of a section of the catheter of FIG. 2;

FIG. 4 shows the catheter of FIG. 2 with a release wire within a release wire lumen;

FIG. 5 is an enlarged overall view of a section of the catheter and release wire of FIG. 4;

FIG. 6 shows the structure of FIG. 5 with a generally helical covered stent mounted thereto in a radially contracted, first state, the structures constituting the distal portion of the coiled stent delivery assembly of FIG. 1, the target location within the body lumen being indicated by dashed lines;

FIG. 6A is an enlarged view of the covered stent of FIG. 6 showing the release wire piercing the distal end of the covered stent;

FIG. 7 illustrates the assembly of FIG. 6 after the release wire has begun to be retracted to release the distal portion of the stent from the catheter;

FIG. 8 shows the assembly of FIG. 7 after the release wire has been retracted further to release part of the intermediate portion of the stent;

FIG. 9 shows the assembly of FIG. 8 after the release wire has been completely retracted and the covered stent is in a radially expanded, second state;

FIG. 10 shows the covered stent of FIG. 9 within the blood vessel and after the catheter has been removed;

FIG. 11 illustrates an alternative embodiment similar to the catheter of FIG. 3 in which the catheter comprises separate tubes connected to one another;

FIG. 12 is an end of view of the catheter of FIG. 11;

FIG. 13 is another alternative embodiment similar to the catheter of FIG. 3 in which the catheter lacks the cutouts of the FIG. 3 embodiment but rather has perforations extending into the release wire lumen, the perforations acting as the entrances and exits of the lumen segments;

FIG. 14 illustrates the embodiment of FIG. 13 in which the release wire passes through the perforations in a weaving pattern;

FIG. 15 illustrates a still further alternative embodiment including a modified release wire assembly similar to the release wire assembly of FIG. 1A and a modified catheter similar to the catheter of FIG. 5;

FIGS. 16 and 17 are cross-sectional views of the catheter taken along lines 16-16 and 17-17 in FIG. 15 illustrating the presence of a distal release wire in FIG. 16 and both the distal and a proximal release wire in FIG. 17;

FIG. 18 is an overall view of the release wire assembly of FIG. 15 showing the use of distal and proximal release wires;

FIG. 18 illustrates the result of initially pulling on the release wire assembly of FIG. 15 causing the proximal release wire to disengage from the proximal end of the covered stent; and

FIG. 20 illustrates the result of continuing to pull on the release wire assembly of FIG. 19 causing the distal release wire to disengage from the distal end of the covered stent, after which continued pulling on the release wire assembly will cause the distal release wire to release the intermediate portion of the covered stent to assume the radially expanded, second state of FIGS. 9 and 10.

### DETAILED DESCRIPTION OF THE INVENTION

The present invention will be described with reference to several embodiments with like reference numerals referring to like elements.

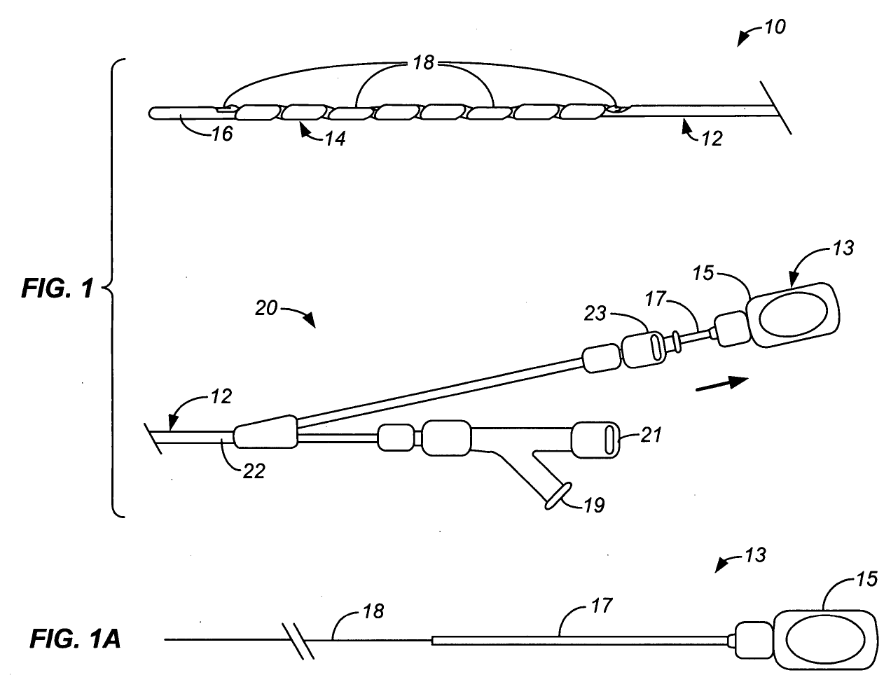

FIG. 1 is an overall view of a coiled stent delivery assembly 10 made according to the invention. Assembly 10 is shown to include a catheter 12 with a generally helical covered stent 14 mounted to the distal portion 16 of catheter 12. Covered stent 14 may be of the type disclosed in U.S. Pat. No. 6,572,648 or 6,645,237 including a ladder type stent covered by a graft material. Assembly 10 also includes a release wire assembly 13, see FIG. 1A, including a flexible release wire 18, extending along catheter 12, connected to a finger grip 15 by a relatively rigid tube 17. Assembly 10 further includes a proximal end assembly 20 secured to the proximal end 22 of catheter 12. Proximal and assembly 20 includes various fittings and ports, such as flush port 19, guide wire port 21 and release wire port 23. The construction of the various components of assembly 10 will now be described.

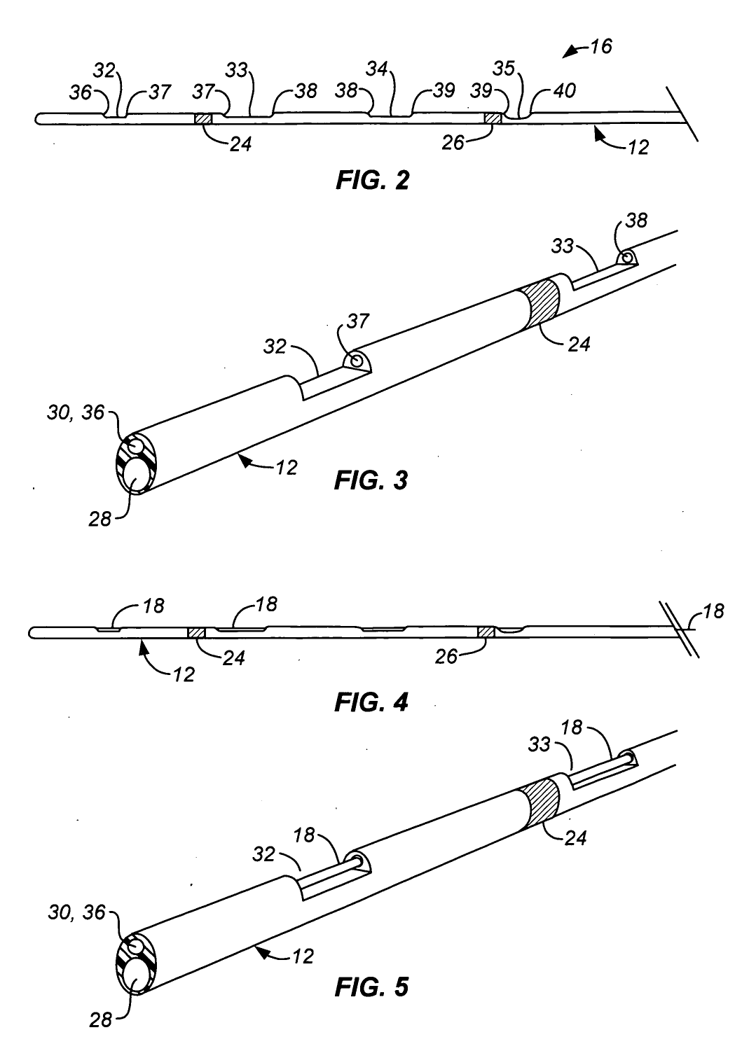

FIG. 2 is a side view of the distal portion 16 of catheter 10 while FIG. 3 is an enlarged overall view of a section of distal portion 16. Distal portion 16 has a pair of radiopaque markers 24, 26 used to help properly position covered sent 14 within the body lumen. Catheter 12 has a main lumen 28 and a release wire lumen 30. Main lumen 28 is typically used for passage of a guide wire and may also be used for other purposes, such as irrigation, aspiration, passage of tissue extraction devices, and so forth. FIGS. 4 and 5 show release wire 18 within release wire lumen 30.

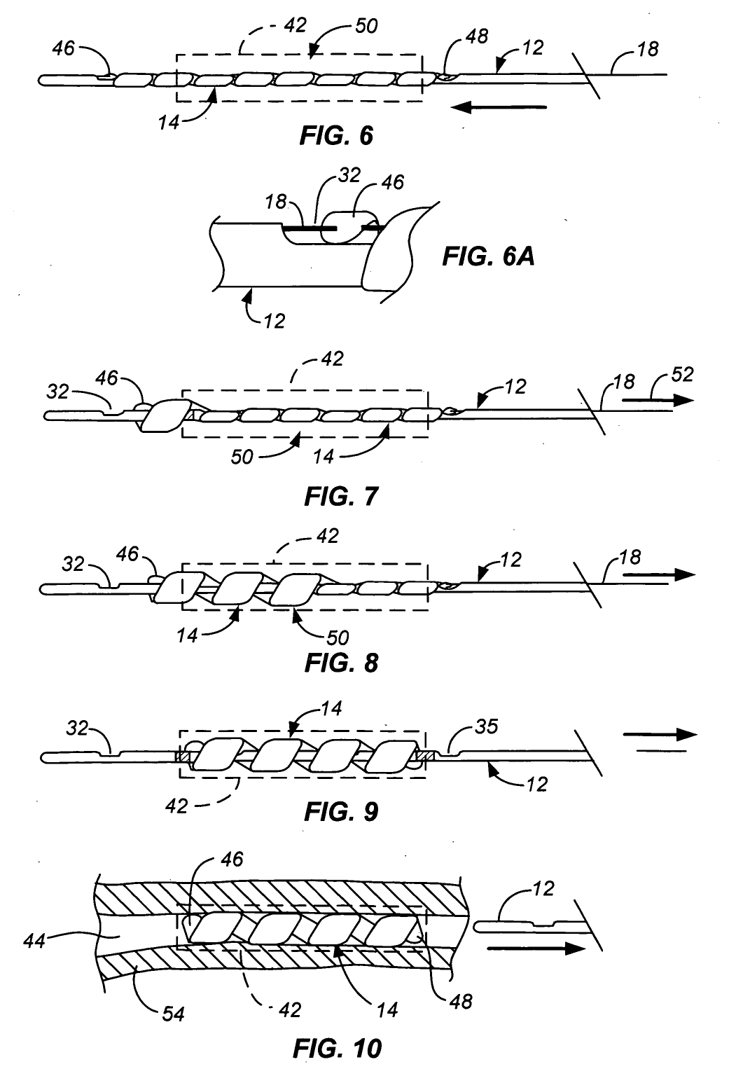

Distal portion 16 of catheter 12 has a number of axially spaced apart cutouts 32, 33, 34 and 35, which create a series of lumen segments 36, 37, 38, 39 and 40 separated by cutouts 32-35. Accordingly, release wire 18 passes along a release wire path including internal release wire path segments defined by lumen segments 36-40 and external release wire path segments along cutouts 32-35. That is, the external release wire path segments extend between the exit of one lumen segment and the entrance of an adjacent lumen segment. FIG. 6 shows covered stent 14 mounted to distal portion 16 of catheter 12 in a radially contracted, first state. Note that covered stent 14 is considered positioned within a body lumen 44 defined by a blood vessel 54, or other hollow body structure, in FIGS. 6-10 but, for simplicity of illustration, blood vessel 54 is only shown in FIG. 10. The target location 42 within the body lumen 44 of blood vessel 54 is indicated by dashed lines. The distal and proximal ends 46 and 48 of covered sent 14 are secured to distal portion 16 of catheter 12 by release wire 18 passing through distal end 46 at cutout 32 as shown in FIG. 6A and through proximal end 48 at cutout 35. The intermediate portion 50 of covered stent 14 is secured to distal portion 16 of catheter 12 at two positions, that is at intermediate cutouts 33, 34, by passing between release wire 18 and catheter 12 and the intermediate cutouts.

The longitudinal or axial length of cutouts 32-35 is oversized with respect to covered stent 14 housed therein. It has been found that making cutouts 32-35, and especially intermediate cutouts 33 and 34, oversized helps to prevent damage to covered stent 14 during assembly and use. In one embodiment catheter 12 has an outside diameter of 1.5 mm (0.060 in.), main lumen 28 has an inside diameter of 1 mm (0.037 in.), release wire 18 has a diameter of 0.3 mm (0.012 in.), and each turn of covered stent 14 when wrapped down as shown in FIG. 1 has an axial or longitudinal length of about 5 or 6 mm. It has been found that making cutouts 33 and 34 about 8 mm, that is about 2 mm longer than the axial or longitudinal length of covered stent 14, helps to eliminate binding of and damage to the covered stent The proximal and distal cutouts 35 and 32 are, in one embodiment, each about 4 mm long. The space between the cutouts is, in this embodiment, 11 mm long to permit two turns of covered stent 14 between each cutout. Some overlap of the turns of covered sent 14 over adjacent cutouts does not compromise the functionality of the catheter. Cutouts 32-35 are shown in FIG. 3 having a flat bottom; the cutouts may also be made with, for example, a convex bottom surface.

Assembly 10 is positioned at target location 42 while in the wound down, radially contracted, first state of FIGS. 1 and 6. Proper positioning of covered stent 14 within blood vessel 54 is aided by the use of radiopaque markers 24, 26. FIG. 7 illustrates release of distal end 46 of covered sent 14 by pulling on release wire 18 as indicated by arrow 52. An undesirable, uncontrolled expansion of covered stent 14, sometimes referred to as a “jack-in-the-box” release, could occur on release of one of the distal and proximal ends 46, 48 of covered stent 14 if it were only secured at its ends. Such a “jack-in-the-box” release is not desired because it can adversely affect the proper final positioning of covered stent 14. A “jack-in-the-box” release is avoided in this embodiment by the provision of intermediate cutout 33, 34 to permit covered stent 14 to be placed between release wire 18 and catheter 12 at the intermediate cutouts.

FIG. 8 illustrates the result of continuing to pull release wire 18 causing release wire 18 to be removed from intermediate cutout 33 thereby releasing a part of intermediate portion 50. FIG. 9 illustrates covered stent 14 within its radially expanded, second state after release wire 18 has been completely removed from catheter 12, that is after removing release wire 18 from intermediate cutout 34 and from proximal end 48 of covered stent 14 at proximal cutout 35. The present invention provides a very controlled release of covered stent 14 to help ensure its proper placement within body lumen 44. FIG. 10 shows covered stent 14 fully expanded within body lumen 44 of blood vessel 54 and after catheter 12 has been removed from blood vessel 54.

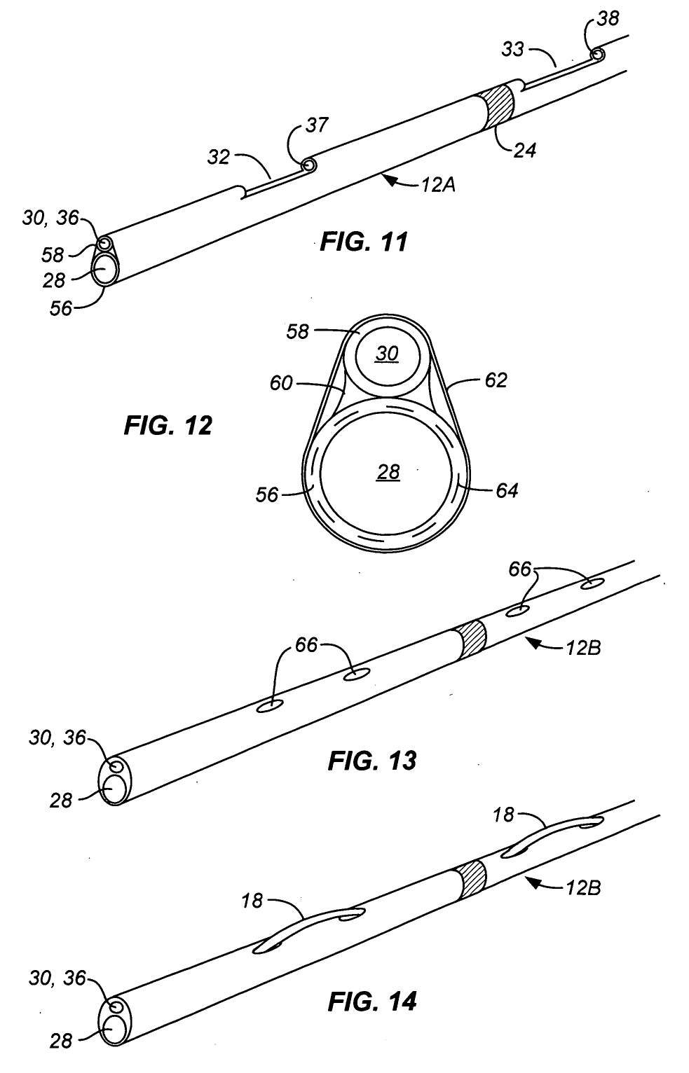

FIGS. 11 and 12 illustrate an alternative embodiment similar to catheter 12 of FIG. 3. Catheter 12A comprises first and second tubes 56, 58 connected to one another by adhesive 60 and heat shrink tubing 62. First tube 56 preferably has stainless steel flat wire braid filaments 64 to enhance structural integrity.

Another alternative embodiment, similar to the catheter of FIG. 3, is shown in FIG. 13. Catheter 12B lacks the cutouts 32-35 of the catheter of FIG. 3 but rather has perforations 66 extending into release wire lumen 30, the perforations acting as the entrances and exits of the lumen segments. FIG. 14 shows catheter 12B with release wire 18 passing through perforations 66 in a weaving pattern so that perforations 66 act as the entrances and exits of lumen segments 36, 37 and 38 in this figure. In some embodiments it may be desired to provide for a much greater number of perforation 66 than would be expected to be used, for example 20 or 40 perforations instead of 8. This would allow greater flexibility in the placement of the turns of covered stent 14 as well as the number of turns to be captured between release wire 18 and sent 12. If the catheter were made from a porous material, the pores in the material itself may provide the perforations. Also, the perforations could also be formed in the catheter using a tool as the covered stent is wound onto the catheter, such a tool could also used to help to guide the release wire out through or into the newly formed perforation or both out through and into the newly formed perforation.

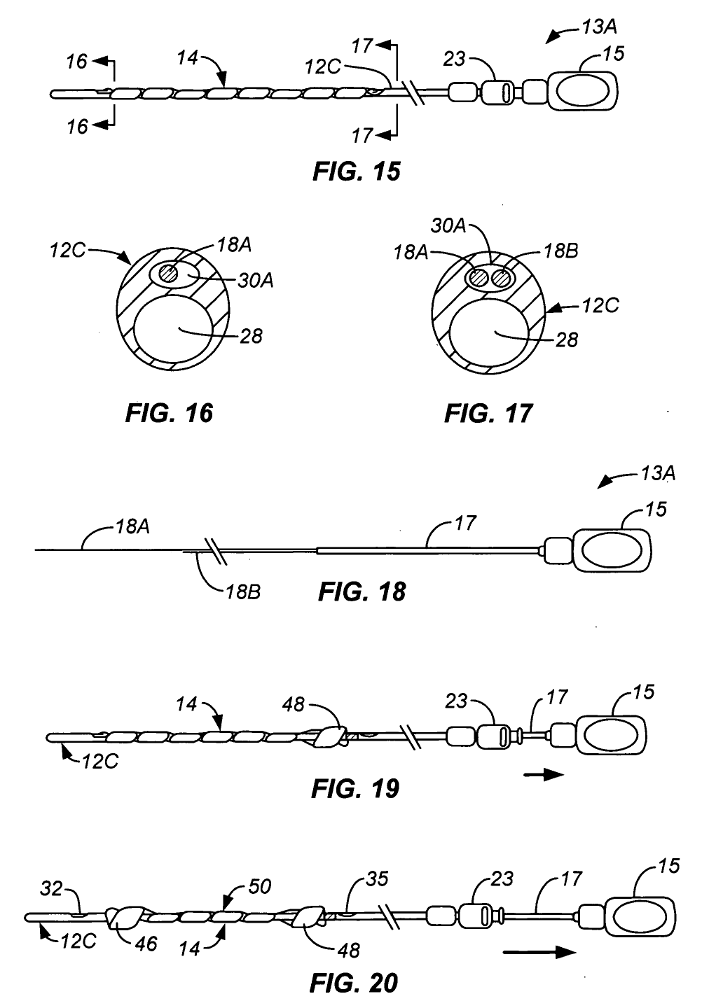

FIG. 15 illustrates aspects of a still further alternative embodiment of the present invention. A modified release wire assembly 13A, shown best in FIG. 18, is used with a modified catheter 12C. FIGS. 16 and 17 are cross-sectional views of catheter 12C showing the presence of an oval or other other-than-round release wire lumen 30A. Release wire lumen 30A is sized and shaped to house both distal and proximal release wires 18A and 18B. If more than two release wires were to be used, the release wire lumen can be appropriately sized and shaped. Also, and may be desirable to use an other-than-round cross-sectional shape for the release wire for greater space utilization.

FIG. 18 is an overall view of the release wire assembly of FIG. 15 showing the use of distal and proximal release wires 18A and 18B. In this embodiment the release wires are pulled simultaneously by finger grip 15. If desired, release wires could be manipulated individually. FIG. 19 illustrates the result of initially pulling on the release wire assembly of FIG. 15. In this embodiment the length of proximal release wire 18B is chosen so that proximal end 48 of covered sent 14 is released first. FIG. 20 illustrates the result of continuing to pull on release wire assembly 13A causing distal release wire 18A to disengage from distal end 46 of the covered stent. Continued pulling on release wire assembly 13A will cause distal release wire 18A to release intermediate portion 50 of covered stent 14 to assume the radially expanded, second state of FIGS. 9 and 10. Instead of using individual release wires, such as distal and proximal release wires 18A and 18B, a single, main release wire can be used having release wire side branches welded or otherwise secured to the main release wire; the release side wire branches would then be the used to engage covered stent 14 and various positions along the covered stent. The lengths of the release wire side branches and the main release wire can be chosen to permit release of covered stent 14 from cutouts 32-35 in any order desired, including from proximal cutout 35 to intermediate cutout 34, to intermediate cutout 33 and finally to distal cutout 32. Such a release from the proximal cutout 35 to the distal cutout 32 could also be accomplished in other manners, such as my extending the release wire to the distal end of the catheter, reversing direction, and then directing the release wire along the release wire lumen back towards the proximal end of the catheter.

Instead of the release schemes discussed above, other release schemes can be used. For example, release can start simultaneously at proximal end 48 and end at distal end 46; also, release of covered stent 14 can be from one or both of intermediate cutouts 34 and then from one end and then from the other end. The number and spacing of the cutouts and perforations can also be changed. Whatever release scheme is to be used, in some embodiments it is preferred that at most 50%, and more preferably at most 25%, of covered sent 14 simultaneously move to the radially expanded, second state in contact with blood vessel 54 or other hollow body structure. In one preferred embodiment, using 4 equally spaced cutouts, at most about 33% of the length of covered stent moves simultaneously to the radially expanded, second state.

The present invention has been described as using a release wire. The release wire is not limited to structures or materials which are commonly classified as wire, that is single or multiple strands of metal. Rather, release wire also includes threads or strands or other lengths of material which may or may not have significant flexural strength and may be nonmetallic or a combination of metallic and nonmetallic materials. The particular mechanical characteristics for the release wire will depend on the operating conditions, including, for example, the length of the cutouts, the force expected to be exerted by the covered stent when in the radially contracted, first state, and the number of release wires used.

The release wire and the associated release wire lumen and lumen openings in the catheter are used to engage the covered stent and maintain it in the radially contracted, first state and then control the subsequent releasing of various portions of the covered stent to prevent the sudden, undesirable “jack-in-the-box” deployment of the covered stent. Instead of a release wire, the covered stent may be retained in the radially contracted, first state using a heat softenable adhesive between the covered stent and the catheter. An appropriate source of heat can be used to selectively heat and thus soften the adhesive. The source of heat could be an RF device positionable at various locations along the main lumen or a number of individually operable resistance heating elements formed in the catheter. Another alternative to a release wire would be to tie the covered stent to the catheter using a loop of thread at each securement point; the loop of thread would pass into the main lumen, through the wall of the catheter, over or through the covered stent, back through the wall of the catheter and into the main lumen to complete the loop. The covered stent could then be released by withdrawing a thread cutter through the main lumen of the catheter causing the loops of thread to be cut, typically one at a time. Other structure and procedures may be used as a substitute for the disclosed release wire arrangement.

Various embodiments of the invention may and preferably do provide one or more of the following advantages: simplicity of design and ease-of-use, ability to release a coiled stent gradually, and accuracy of placement.

Other modification and variation can be made to the disclosed embodiments without departing from the subject of the invention as defined in following claims. For example, instead of providing a separate release wire lumen, in some embodiments the delivery catheter may include a single lumen through which the release wire passes; however, it is preferred that a separate release wire lumen be provided because having a separate release wire lumen helps to reduce the tendency of the release wire to bend so the release wire holds the covered stent more securely. Having a separate release wire lumen helps to prevent any interference with the passage of the guide wire or other devices through the catheter. In some situations it may not be necessary to provide distal lumen segment 36. For example, the distal end of release wire 18 could be releasably secured to distal end 46 of covered stent 14 by, for example, bending the distal end of the release wire (which would straighten when pulled), adhering the release wire to the distal end using an adhesive (which adhesive bond could be broken when the release wire was pulled), or a securing the release wire to the distal end by a breakable thread (which would break when the release wire was pulled). In the preferred embodiments the release wire engages the tips of the proximal and distal portions of the covered stent; in appropriate cases it may be possible or desirable to engage the covered stent at positions spaced apart from the tips of the proximal and distal portions. The invention has been described with reference to a covered stent. However, other generally helical endoluminal prostheses may also be used. For example, a bare metal stent or a metal stent coated with a polymer/drug matrix may be used. In the preferred embodiments the release wire passes through or pierces the proximal and distal ends of the covered stent while the intermediate portion of the covered stent passes between the release wire and the catheter; in some situations it may be desirable to have the release wire pierce one or more locations along the intermediate portion of the covered stent. While the stent is typically released by pulling on the release wire, release may also be accomplished in appropriate situations by pushing on the release wire.

Any and all patents, patent applications and printed publications referred to above are incorporated by reference.

What is claimed is:

1. A coiled endoluminal prosthesis delivery assembly comprising:
a catheter,
a generally helical endoluminal prosthesis having proximal and distal portions separated by an intermediate portion, the endoluninal prosthesis being placeable in a radially contracted, first state on the catheter;
means for engaging each of the proximal, intermediate and distal portions thereby maintaining the endoluminal prosthesis in the first state; and
means for controllably releasing the proximal, distal and intermediate portions to permit the endoluminal prosthesis to move towards a radially expanded, second state.

2. The assembly according to claim 1 wherein the controllably releasing means comprises means for releasing the portions one at a time.

3. The assembly according to claim 2 wherein the releasing means comprises means for releasing the portions in order from a chosen one of the proximal and distal ends towards the other of the proximal and distal ends.

4. The assembly according to claim 1 wherein the controllably releasing means comprises means for releasing the intermediate portion after the proximal and distal portions.

5. The assembly according to claim 4 wherein the controllably the releasing means comprises means for simultaneously releasing the proximal and distal portions.

6. The assembly according to claim 1 wherein the engaging means comprises a release wire, the intermediate portion of the endoluminal prosthesis passing between the release wire and the catheter at least one position along the intermediate portion.

7. A coiled endoluminal prosthesis delivery assembly comprising:
a catheter comprising an outer surface, a lumen, and a number of openings extending from the outer surface to the lumen;
a release wire extending along a release wire path;
the release wire path comprising internal release wire path segments defined within the lumen and external release wire path segments external of the catheter, and
a generally helical endoluminal prosthesis having proximal and distal portions separated by an intermediate portion, the endoluninal prosthesis being maintained in a radially contracted condition by the release wire engaging the proximal, distal and intermediate portions of the endoluminal prosthesis at least three of the external release wire path segments;
whereby the coiled endoluminal prosthesis is releasable from the catheter when the release wire is moved along the release wire path.

8. The assembly according to claim 7 wherein the outer surface is an interrupted surface comprising a number of recessed surfaces with at least some of the openings extending from the recessed surfaces to the lumen.

9. The assembly according to claim 8 wherein the recessed surfaces are sized for housing a portion of a turn of the endoluminal prosthesis.

10. The assembly according to claim 9 wherein the recessed surfaces have a longitudinal length oversized with respect to the portion of the turn of the endoluminal prosthesis housed therein.

11. The assembly according to claim 10 wherein the longitudinal length of the recessed surfaces are about 2 mm oversized with respect to the endoluminal prosthesis housed therein.

12. The assembly according to claim 8 wherein the recessed surfaces comprise cutout surfaces.

13. The assembly according to claim 11 wherein the outer surface is a generally uninterrupted surface so that the release wire path comprises a woven release wire path.

14. A coiled endoluminal prosthesis delivery assembly comprising:
a catheter comprising a release wire lumen, the release wire lumen comprising a series of axially spaced-apart lumen segments, each lumen segment comprising an entrance and exit;
a release wire extending along a release wire path;
the release wire path comprising internal release wire path segments defined by the lumen segments and external release wire path segments extending between the exit of one lumen segment and the entrance of another lumen segment;
a generally helical endoluminal prosthesis having proximal and distal portions separated by an intermediate portion, the endoluminal prosthesis being maintained in a radially contracted condition with:
the intermediate portion passing between the catheter and the release wire at at least one of the external release wire path segments; and
the proximal and distal portions of the endoluminal prosthesis having release wire engagement parts with which the release wire releasably engages;
whereby the coiled endoluminal prosthesis is releasable from the catheter when the release wire is moved along the release wire path.

15. A coiled endoluminal prosthesis delivery assembly comprising:
a catheter comprising an outer surface, a main lumen, a release wire lumen, and a number of openings extending from the outer surface to the release wire lumen;
a release wire extending along a release wire path;
the release wire path comprising internal release wire path segments defined within the release wire lumen and external release wire path segments external of the catheter; and
a generally helical endoluminal prosthesis having proximal and distal portions separated by an intermediate portion, the endoluminal prosthesis being maintained in a radially contracted condition by the release wire engaging the endoluminal prosthesis at at least two of the external release wire path segments;
whereby the coiled endoluminal prosthesis is releasable from the catheter when the release wire is moved along the release wire path.

16. The assembly according to claim 15 wherein the release wire lumen has an other-than-circular cross-sectional shape.

17. The assembly according to claim 16 wherein the release wire comprises first and second release wire segments engaging different portions of the endoluminal prosthesis.

18. The assembly according to claim 15 wherein the catheter comprises a first tube defining the main lumen and a second tube defining the release wire lumen.

20. A method for controllably releasing a generally helical endoluminal prosthesis from a catheter within a body lumen of a hollow body structure, the endoluminal prosthesis being mounted onto the catheter in a radially contracted, first state, comprising:
placing the endoluminal prosthesis, carried by the catheter, at a target location within a body lumen, the endoluminal prosthesis comprising proximal, distal and intermediate portions, each of which is temporarily retained in the radially contracted, first state;
releasing one or more of the distal, intermediate and proximal portions to move towards a radially expanded, second state in contact with the hollow body structure while maintaining the unreleased portions in the radially contracted, first state;
the releasing step comprising permitting a portion of the endoluminal prosthesis to move to the radially expanded, second state in contact with the hollow body structure;
thereafter selectively releasing the remaining one or ones of the proximal, distal and intermediate portions to permit the entire endoluminal prosthesis to move to the radially expanded, second state in contact with the hollow body structure; and
removing the catheter from the body lumen.

20. The method of according to claim 19 wherein the releasing and selectively releasing steps are carried out to release all portions of the endoluminal prosthesis one at a time.

21. The method of according to claim 19 wherein the releasing and selectively releasing steps are carried out to release the distal portion a chosen one of first, second and last.

22. The method of according to claim 21 wherein the releasing and selectively releasing steps are carried out to release the at least part of the intermediate portion second.

23. The method of according to claim 21 wherein the releasing and selectively releasing steps are carried out to release the proximal portion a chosen one of first, second and last.

24. The method of according to claim 19 wherein the releasing and selectively releasing steps are carried out to simultaneously release two or more of the distal portion, proximal portion and a part of the intermediate portion.

25. The method of according to claim 19 wherein the releasing step comprises permitting at most 50% of the length of the endoluminal prosthesis to simultaneously move to the radially expanded, second state in contact with the hollow body structure.

26. The method of according to claim 19 wherein the releasing step comprises permitting at most 25% of the length of the endoluminal prosthesis to simultaneously move to the radially expanded, second state in contact with the hollow body structure.

27. The method of according to claim 19 wherein the releasing step is carried out to release:
one or both of the distal and proximal portions to move towards a radially expanded, second state while maintaining the unreleased portions in the radially contracted, first state; or
at least a part of the intermediate central portion to move towards a radially expanded, second state while maintaining the unreleased portions in the radially contracted, first state.
